(19) United States
(12) Patent Application Publication
(10) Pub. No.: US 2024/0265997 A1
(43) Pub. Date: Aug. 8, 2024

(54) DISEASE OR DRUG ASSOCIATION PROVIDING SYSTEM FOR DIGITAL TWINS WITH GENETIC INFORMATION SCREENED BY ARTIFICIAL INTELLIGENCE

(71) Applicant: PREDICTIV CARE, INC., Mountain View, CA (US)

(72) Inventor: Sijung YUN, Mountain View, CA (US)

(21) Appl. No.: 18/298,614

(22) Filed: Apr. 11, 2023

Related U.S. Application Data
(63) Continuation of application No. PCT/US2023/065398, filed on Apr. 5, 2023.

(30) Foreign Application Priority Data
Feb. 7, 2023 (KR) 10-2023-0016383

(51) Int. Cl. G16B 25/10; G16B 35/20; G16H 10/60; G16H 20/10; G16H 50/30

(52) U.S. Cl. CPC G16B 25/10; G16B 35/20; G16H 10/60; G16H 20/10; G16H 50/30

(57) ABSTRACT

The present disclosure relates to a disease or drug association providing system for a digital twin with genetic information screened by an artificial intelligence. To this end, provided is the disease or drug association providing system for a digital twin with genetic information screened by an artificial intelligence, the system including, a clinical information generating unit including, (i-1) a genetic informatics pipeline unit which sequentially outputs genetic sequencing data of a patient in a predetermined unit, (i-2) a predicted candidate group screening AI unit which screens a predicted candidate group for the disease and a predicted candidate group for the drug with respect to the genetic information output from the genetic informatics pipeline unit, (i-3) a dimensionality reduction AI unit which reduces a size of the genetic information, and (i-4) a genetic informatics integration unit which receives and integrates the genetic information trained by the predicted candidate group screening AI unit and the dimensionality reduction AI unit from the genetic informatics pipeline unit, (ii) a patient's digital twin file which is virtually generated based on information about a body of the patient, and the predicted candidate group for the disease and the predicted candidate group for the drug received from the genetic informatics integration unit, (iii) a disease information table which receives and stores information about a disease from the outside, (iv) a drug information table which receives and stores information about a drug from the outside, and (v) a controller which calculates and provides an association with the disease information table about the genetic information of the digital twin file and an association with the drug information table about the genetic information of the digital twin file.

### TECHNICAL FIELD

The present disclosure relates to a system for predicting a disease possibility or a risk of a drug of a patient, and more particularly, to a disease or drug association providing system for a digital twin with genetic information screened by an artificial intelligence.

### BACKGROUND ART

Generally, a patient used to cure the disease by directly taking the medicine prescribed by a doctor. Accordingly, even though a patient has a side effect to a specific medicine due to the physical constitution, diseases, health condition, and age of the patient, the side effect may be confirmed only by taking the medicine. That is, the patient has to accept the anxiety and the risk of a medicine to be taken for the first time.

Further, even for the medicine which is widely generalized and used (for example, Tylenol, and Aspirin), the side effects which may occur with a low probability cannot be avoided. Patients who experience the rare side effects will inevitably damage their health by taking the medicine. Recently, in order to minimize the drug side effects, services are provided to check and guide concomitant prescription or concurrent medication in advance. However, these services had limitation to be provided based on only the chemical reaction between drugs by ignoring the characteristic of an individual patient.

Further, it is known that a specific drug does not respond well to a patient with a certain genetic variant. Despite such academic knowledge, the genetic test is not performed on all patients before administration, and the test is practically impossible.

In the meantime, in some cases, genetic causes or family history may cause a high possibility of specific diseases. For example, obesity genes may cause an adult disease, certain cancer diseases, or glaucoma. In this case, even though a current health condition of the patient is good, consistence management and observation are requested.

### DISCLOSURE

### Technical Problem

Accordingly, the present disclosure has been made an effort to solve the above-described problems, and an object to be achieved by the present disclosure is to provide a disease or drug association providing system for a digital twin with genetic information screened by an artificial intelligence which implements a digital twin by coupling a patient's gene and a physical characteristic.

Another object of the present disclosure is to provide a disease or drug association providing system for a digital twin with genetic information screened by an artificial intelligence which may predict the individually susceptible disease and/or potential drug side effect in advance by applying a next generation sequencing (NGS) technique to a patient's gene and coupling individual physical characteristics to configure a genome of the patient in a digital form.

Technical objects to be achieved in the present disclosure are not limited to the aforementioned technical objects, and another not-mentioned technical object will be clearly understood by those skilled in the art from the description below.

### Technical Solution

In order to achieve the above-described technical objects, provided is a disease or drug association providing system for a digital twin with genetic information screened by an artificial intelligence, the system including, a clinical information generating unit including: (i-1) a genetic informatics pipeline unit which sequentially outputs genetic sequencing data of a patient in a predetermined unit; (i-2) a predicted candidate group screening AI unit which screens a predicted candidate group for the disease and a predicted candidate group for the drug with respect to the genetic information output from the genetic informatics pipeline unit; (i-3) a dimensionality reduction AI unit which reduces a size of the genetic information; and (i-4) a genetic informatics integration unit which receives and integrates the genetic information trained by the predicted candidate group screening AI unit and the dimensionality reduction AI unit from the genetic informatics pipeline unit; (ii) a patient's digital twin file which is virtually generated based on information about a body of the patient, and the predicted candidate group for the disease and the predicted candidate group for the drug received from the genetic informatics integration unit; (iii) a disease information table which receives and stores information about a disease from the outside; (iv) a drug information table which receives and stores information about a drug from the outside; and (v) a controller which calculates and provides an association with the disease information table about the genetic information of the digital twin file and an association with the drug information table about the genetic information of the digital twin file.

Further, the association of the digital twin file with the disease information table for the genetic information is calculated based on a variant gene name and a disease name among the genetic information.

Further, the association of the digital twin file with the drug information table for the genetic information is calculated based on the variant gene name and a drug name among the genetic information.

Further, the association of the digital twin file with the disease information table for the genetic information or the association of the digital twin file with the drug information table for the genetic information calculated by the controller may be provided to an external service unit via a network.

Further, the service unit is an external application program of a client terminal which is connected to the network or a dedicated application program which is directly connected to the controller via the network.

Further, the external application program or the dedicated application program may output a report including an association of the digital twin file with disease information table for genetic information and an association of the digital twin file with a drug information table for genetic information.

Further, the report further includes a detailed disease informatics unit for the disease and a detailed drug informatics unit for the drug.

Further, the digital twin file further includes a 3D human body avatar model.

a body part for the disease having the highest association or a body part for the drug having the highest association is graphically displayed on the 3D human body avatar model.

Further, the dimensionality reduction AI unit reduces the genetic information in a range of 20000.

### Advantageous Effects

According to the exemplary embodiment of the present disclosure, a patient provides genes from the fingernail or hair so that once the digital twin file of the patient is generated, the disease possibility and the drug side effect may be consistently notified through the digital twin later. Accordingly, there is a convenience that the patient does not need to annoyingly repeat the gene test.

According to the present disclosure, a susceptible disease which is highly likely to occur in the future may be notified in advance. By doing this, individual may seek to improve dietary life, regular exercise, and change in lifestyle according to their individual characteristics, and may respond early in the event of an onset through constant examination of the disease.

Further, the patient may be notified in advance of the possibility of the drug side effects through the digital twin of the patient. Specifically, even though it is a widely used drug, the possibility of ineffectiveness or side effects depending on the characteristics of the patient's genes may be blocked in advance.

Accordingly, the digital twin system may be easily referenced in real time during the doctor's treatment or prescription to significantly reduce the medical risks or medical accidents.

A technical object to be achieved in the present disclosure is not limited to the aforementioned effects, and another not-mentioned effects will be obviously understood by those skilled in the art from the description below.

### DESCRIPTION OF DRAWINGS

The following drawings attached to the present specification illustrate the exemplary embodiment of the present disclosure and are provided for better understanding of the technical spirit of the present disclosure together with the detailed description of the disclosure to be described. Therefore, the present disclosure should not be construed as being limited only to the matters described in the drawings.

FIG. 1 is a diagram illustrating a login screen 10 to access a system from an external service unit according to an example embodiment of the present disclosure.

FIG. 2 is a diagram illustrating a dashboard screen 20 of a plurality of patients provided by a system according to an example embodiment of the present disclosure.

FIG. 3 is a diagram illustrating a patient information screen 40 displayed by a patient icon 32 of a system according to an example embodiment of the present disclosure.

FIG. 4 is a diagram illustrating a main screen 500 displayed by a digital twin icon 34 of a system according to an example embodiment of the present disclosure.

FIG. 5 is a diagram illustrating a susceptible disease predicting unit 800 displayed by a susceptible disease icon 580 of a system according to an example embodiment of the present disclosure.

FIG. 6 is a diagram illustrating a detailed disease informatics unit 850 displayed by clicking one disease of FIG. 5 according to an example embodiment of the present disclosure.

FIG. 7 is a diagram illustrating a drug risk predicting unit 900 displayed by a drug icon 585 of a system according to an example embodiment of the present disclosure.

FIG. 8 is a diagram illustrating a detailed drug informatics unit 1000 displayed by clicking one drug of FIG. 7 according to an example embodiment of the present disclosure.

FIG. 9 is a diagram illustrating a genetic variant list 700 displayed by a genetic information icon 590 of a system according to an example embodiment of the present disclosure.

FIG. 10 is a schematic block diagram of a digital twin system according to an example embodiment of the present disclosure.

### BEST MODE

In the following detailed description, only certain exemplary embodiments of the present disclosure have been shown and described, simply by way of illustration. Description of the present disclosure is just an embodiment for structural and functional description so that the scope of the present disclosure is not interpreted to be limited by the embodiment described in the specification. That is, the embodiment may be modified in various forms so that it is understood that the scope of the present disclosure has equivalents which are capable of implementing the technical spirit. Further, it does not mean that the specific embodiment includes the object or effect proposed in the present disclosure or includes only the effect so that it is not understood that the scope of the present disclosure is limited thereby.

In the meantime, meanings of terms described in the present disclosure can be understood as follows.

The terms “first” or “second” are used to distinguish one component from the other component so that the scope should not be limited by these terms. For example, a first component may be referred to as a second component, and similarly, a second component may be referred to as a first component. It should be understood that, when it is described that an element is “connected” to another element, the element may be directly connected to the other element or connected to the other element through a third element. In contrast, it should be understood that, when it is described that an element is directly connected to another element, no element is present between the element and the other element. Other expressions which describe the relationship between components, that is, “between” and “directly between”, or “adjacent to” and “directly adjacent to” need to be interpreted by the same manner.

Unless the context apparently indicates otherwise, it should be understood that terms “include” or “have” indicate that a feature, a number, a step, an operation, a component, a part or the combination thereof described in the specification is present, but do not exclude a possibility of presence or addition of one or more other features, numbers, steps, operations, components, parts or combinations thereof, in advance.

Unless they are contrarily defined, all terms used herein including technological or scientific terms have the same meaning as those generally understood by a person with ordinary skill in the art. Terms which are defined in a generally used dictionary should be interpreted to have the same meaning as the meaning in the context of the related art but are not interpreted as an ideally or excessively formal meaning if it is not clearly defined in the present disclosure.

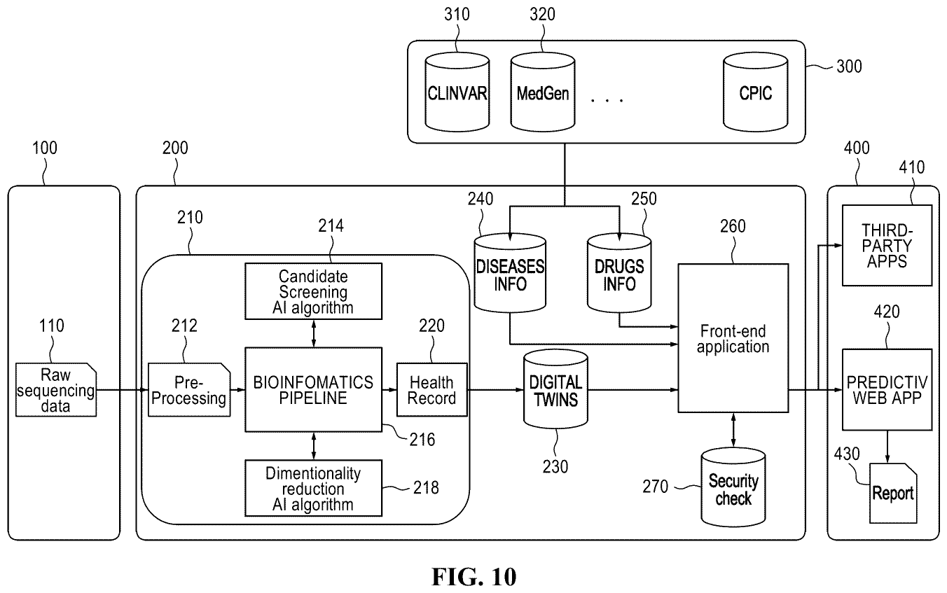

Hereinafter, preferred exemplary embodiments will be described in detail with reference to the accompanying drawings. FIG. 10 is a schematic block diagram of a digital twin system according to an example embodiment of the present disclosure. As illustrated in FIG. 10, a DNA sequencing analysis unit 100 extracts a DNA from a fingernail or hair of a specific patient to generate DNA sequencing data (raw sequencing data) 110.

The digital twin predicting unit 200 generates a digital twin file and provides a service for a medical risk about the disease or the drug side effect to the outside. The digital twin predicting unit 200 may be a server computer connected to a network (for example, internet), and bi-directionally communicate with a public DB 300. The digital twin predicting unit 200 is a platform for a gene-based digital twin system.

A DNA preprocessing unit 212 preprocesses the DNA sequencing data 110 to have a specific format to be computed in a genetic informatics pipeline unit 216. For example, the DNA preprocessing unit 212 applies a next generation sequencing (NGS) technique to an individual DNA and couples a physical characteristic of the individual to configure an individual genome as a digital form.

The genetic informatics pipeline unit 216 modulates the preprocessed DNA sequences in a predetermined unit to sequentially output the preprocessed DNA sequences. The predetermined unit of DNA sequences is transmitted to a predicted candidate group screening AI unit 214 and a dimensionality reduction AI unit 218, and completed data is transmitted to a genetic informatics integration unit 220.

The predicted candidate group screening AI unit 214 is an artificial intelligence model which screens a predicted candidate group for the disease and a predicted candidate group for a drug with respect to the genetic information of the DNA transmitted from the genetic informatics pipeline unit 216. To this end, the predicted candidate group screening AI unit 214 performs machine learning on numerous medical information, clinical information, and genetic information through a neural network and a convolution neural network (CNN). To be more specific, the predicted candidate group screening AI unit 214 performs machine learning on 20000 pieces of genetic information, 22000 pieces of disease information for human and 750 types of drug information approved by the FDA. Further, the predicted candidate group screening AI unit 214 periodically learns latest medical information to enable the reasoning based on the latest information. For example, the predicted candidate group screening AI unit 214 learns and infers that if there is a specific genetic variant, a possibility of onset of a specific disease is high. Further, the predicted candidate group screening AI unit 214 learns and infers that if there is a specific genetic variant, there is a side effect to a specific drug.

The dimensionality reduction AI unit 218 serves to reduce approximately 3 billion pieces of DNA information provided from the bioinformatics pipeline unit 216 to predetermined 20000 pieces of genetic information. The dimensionality reduction AI unit 218 enables the rapid prediction by avoiding unnecessary computations to save a computing resource.

The genetic informatics integration unit 220 receives and integrates genetic information trained by the predicted candidate group screening AI unit 214 and the dimensionality reduction AI unit 218, from the genetic informatics pipeline unit 216. The digital twin 230 which comprehensively represents a characteristic for the patient is generated by the integration described above.

A clinical information generating unit 210 includes, aforementioned, the DNA preprocessing unit 212, the predicted candidate group screening AI unit 214, the genetic informatics pipeline unit 216, the dimensionality reduction AI unit 218, and the genetic informatics integration unit 220. The clinical information generating unit 210 generates a digital twin 230.

The digital twin is a model file in which genetic information and physical characteristic information of the patient are coupled, and the digital twin matches the corresponding patient one to one. The digital twin allows finding a susceptible disease of the patient in advance and identifying a drug side effect in advance. Accordingly, the digital twin may also be called a patient bio information file, a digital pseudo-body, or a virtual patient model.

The public database 300 is a medical database service provided from external institutions. For example, the public data may be CLINVAR 310 which is an open archive providing interpretation of human genetic variation and disease from National Center for Biological Information (NCBI) in the United States, MedGen 320 which provides information related to the human genes, PharmGKB which provides information about influences of the human genetic variation on a drug response, DAILYMED which provides drug information, OpenFDA which summarizes data from the US FDA to provide the data to the public, and CPIC which is provided by the Cancer Prevention Institute of California.

A disease information table 240 is a database table which stores disease related information received from the public database 300.

A drug information table 250 is a database table which stores drug related information received from the public database 300.

A controller 260 reads the digital twin 230, the disease information table 240, and the drug information table 250 to calculate an onset possibility of the susceptible disease and a risk of the drug side effects. The controller 260 is an application program executed in the server computer.

A security checking unit 270 checks the risk of concomitant prescriptions or concurrent medications in advance.

The service unit 400 is an external electronic device which may access the digital twin predicting unit 200 via a network. The network may be internet, LAN, intranet, a dedicated administrative net, and may be a wired or wireless network. The service unit 400 may be a client computer, a laptop computer, a smart phone, or a tablet PC. In the service unit 400, an external application 410 may be executed or a dedicated application 420 may be executed, and a separate report (for example, a printed material or a PDF file) 430 may be output.

Further, the external application 410 program or the dedicated application 420 program may output a report 430 including an association of the digital twin file with disease information table for genetic information and an association of the digital twin file with a drug information table for genetic information. The report 430 further includes a detailed disease informatics unit 850 for a disease and a detailed drug informatics unit 1000 for a drug.

### MODE FOR CARRYING OUT THE INVENTION

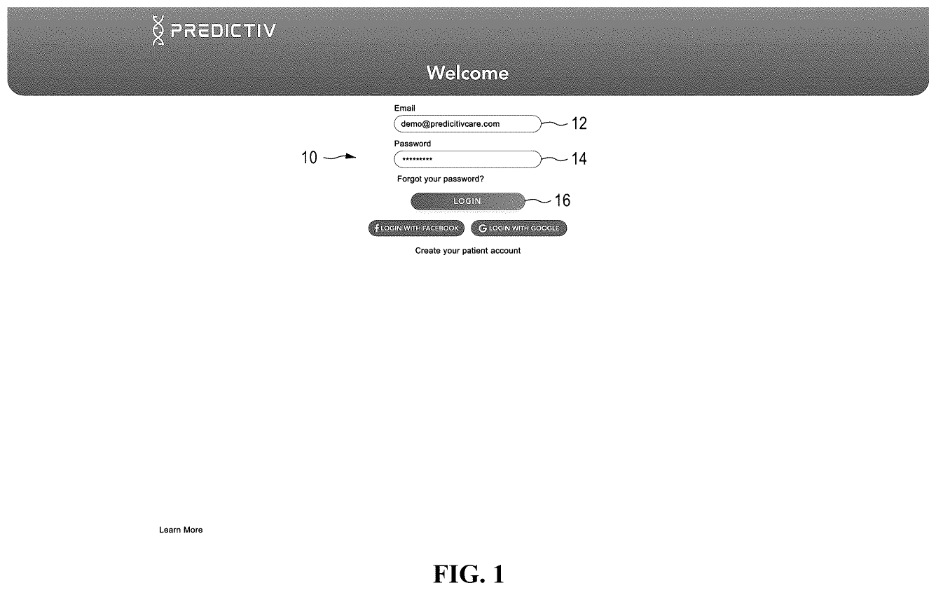

FIG. 1 is a diagram illustrating a login screen 10 to access a system from an external service unit according to an example embodiment of the present disclosure. The external application 410 program of the service unit 400 inputs a specific IP address to access the controller 260. Next, as illustrated in FIG. 1, a user inputs an email address in an email input window 12 and inputs a password in a password input window 14, and then presses the login button 16 to attempt the login. To this end, the digital twin 230 needs to be created in advance by member registration and gene provision.

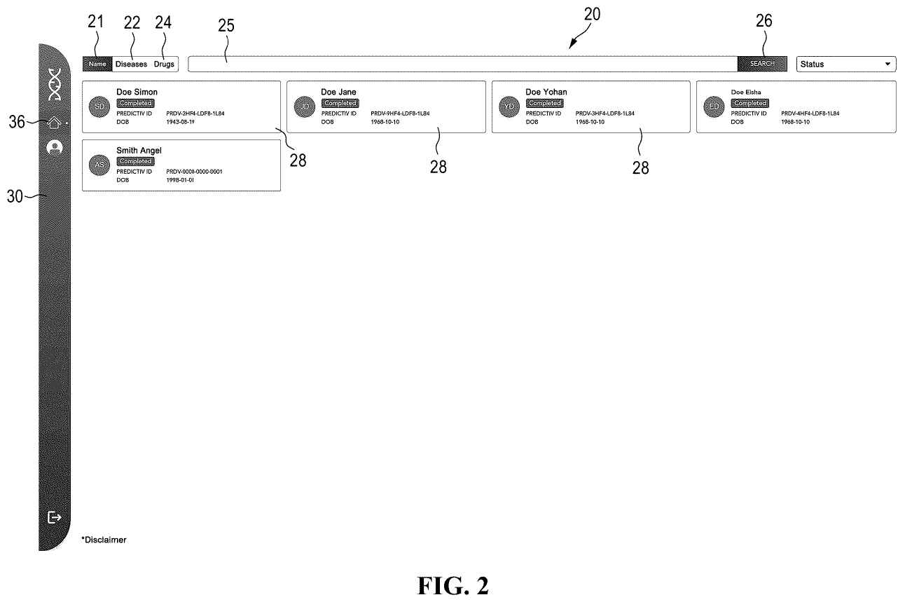

FIG. 2 is a diagram illustrating a dashboard screen 20 of a plurality of patients provided by a system according to an example embodiment of the present disclosure. As illustrated in FIG. 2, a home icon 36 is displayed on a left menu bar 30, and a patient name tab 21, a disease tab 22, and a drug tab 24 are provided at the top. When a search button 26 is pressed after selecting the patient name tab 21 and inputting a part of the patient name in a search keyword input window 25, a search result is displayed in the form of a card. In FIG. 2, all five registered patient names 28 are displayed.

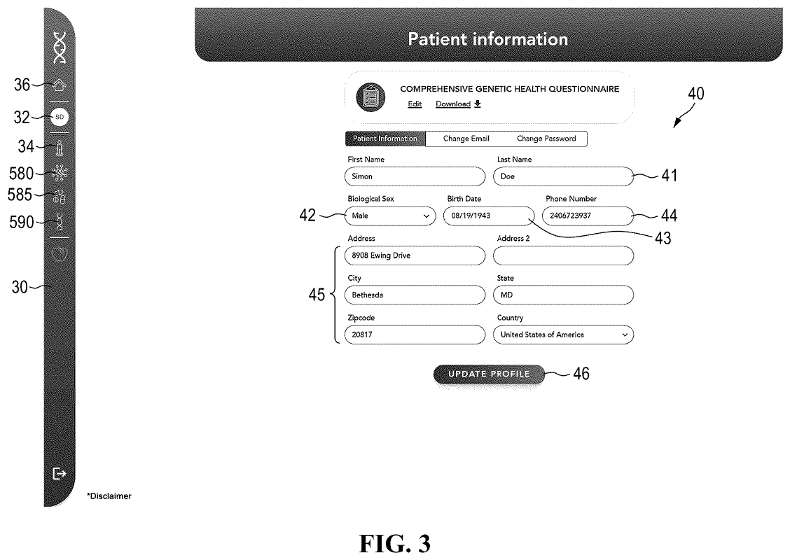

FIG. 3 is a diagram illustrating a patient information screen 40 displayed by a specific patient icon 32 of FIG. 3 according to an example embodiment of the present disclosure. As illustrated in FIG. 3, the menu bar 30 is displayed at the left side. On the menu bar 30, a home icon 36, a patient icon 32, a digital twin icon 34, a susceptible disease icon 580, a drug icon 585, and a genetic information icon 590 are displayed.

In the patient information screen 40, a patient's name 41, a biological sex 42, a birth date 43, a phone number 44, and an address 45 are displayed, which may be modified as needed. When the input button 46 is pressed, information of the patient is input.

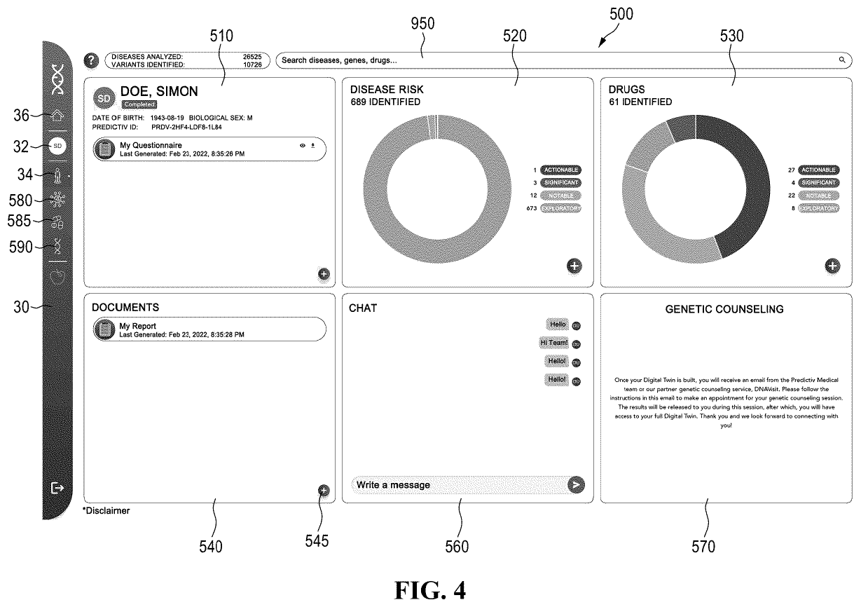

FIG. 4 is a diagram illustrating a main screen 500 displayed by a digital twin icon 34 of a system according to an example embodiment of the present disclosure. As illustrated in FIG. 4, the main screen 500 displays a medical record part 510, a disease prediction part 520, a drug side effect prediction part 530, a document part 540, a chatting part 560, and a memo pad 570.

In the medical record part 510, personal information of the patient (for example, name, a date of birth, and a personal ID) and physical information (for example, a biological sex, a medical history, and a family history) are displayed.

In the disease prediction part 520, a risk of the patient which catches the susceptible disease is displayed on a graph. For example, 680 diseases are reviewed for the patient. Among them, 689 cases of an exploratory level, 12 cases of a notable level, three cases of a significant level, and one case of actionable level having a high onset possibility are inferred by the predicted candidate group screening AI unit 214 to be displayed.

The drug side effect predicting unit 530 predicts and displays the drug side effects of the patient. For example, 61 drugs are reviewed for the patient. Among them, 8 cases of an exploratory level, 22 cases of a notable level, four cases of a significant level, and 27 cases of actionable level having a high side effect possibility are inferred by the predicted candidate group screening AI unit 214 to be displayed.

In a document part 540, a document creation button 545 may be pressed to create and input a separate clinical note.

The chatting unit 560 enables chatting through the message input when the remote medical examination is performed between a patient and a doctor.

The memo pad 570 allows a separate memorandum or a medical appointment.

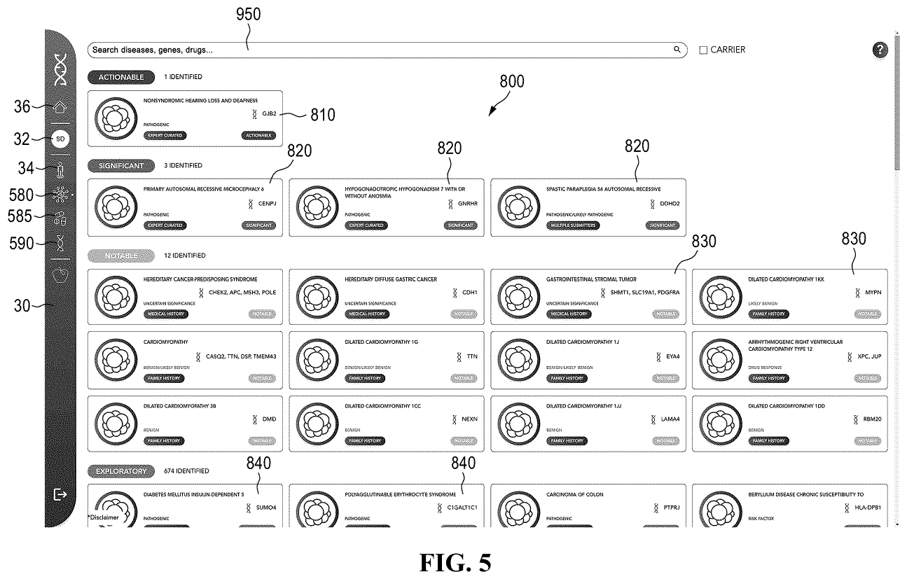

FIG. 5 is a diagram illustrating a susceptible disease predicting unit 800 displayed by a susceptible disease icon 580 of a system according to an example embodiment of the present disclosure. As illustrated in FIG. 5, a first level of susceptible disease 810 is an actionable level having a very high onset possibility and is displayed with red. A second level of susceptible disease 820 is a significant level and is displayed with an orange color. A third level of susceptible disease 830 is a notable level and is displayed with yellow. A fourth level of susceptible disease 840 is an exploratory level and is displayed with gray.

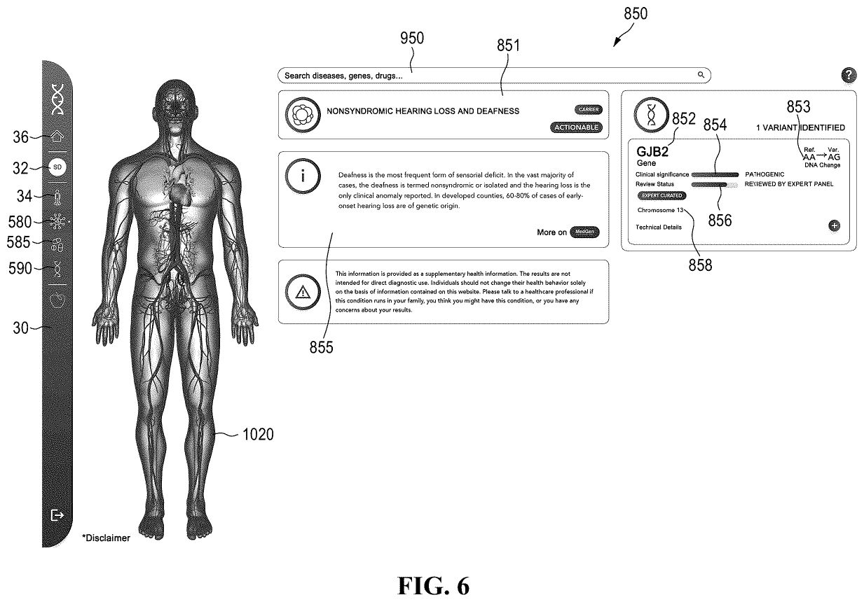

FIG. 6 is a diagram illustrating a detailed disease informatics unit 850 displayed by clicking one disease of FIG. 5 according to an example embodiment of the present disclosure. As illustrated in FIG. 6, a disease name 851 related to the disease, a name of a variant gene 852, a clinical significance 854, disease information 855, a review state 856, and a position and detailed information 858 of the gene are displayed. An integrated search part 950 through which a disease name, a gene name, and a drug name may be comprehensively searched is located at the top. Further, a 3D human body avatar 1020 is three-dimensionally displayed at the left side, and is displayed as a rotating image. Further, on the 3D human body avatar 1020, a site (for example, liver) on which the disease name 851 affects is displayed with the other color or in blinking. The 3D human body avatar 1020 may be used as a graphic model of the digital twin 230.

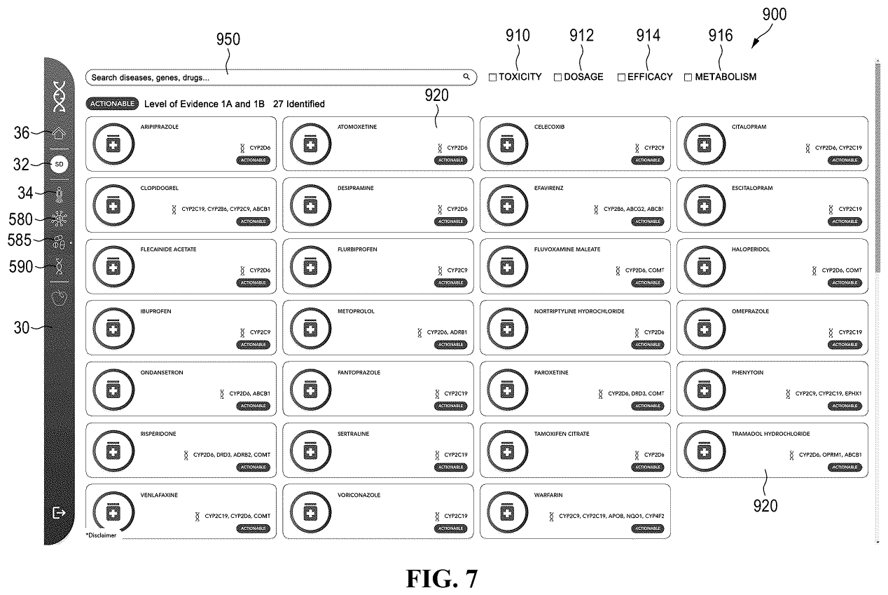

FIG. 7 is a diagram illustrating a drug risk predicting unit 900 displayed by a drug icon 585 of a system according to an example embodiment of the present disclosure. As illustrated in FIG. 7, an integrated search part 950 is provided at the top and the menu bar 30 is provided at the left side. As illustrated in FIG. 7, one of a toxicity 910, a dosage 912, an efficacy 914, and a metabolism 916 of the drug may be selected by a checkbox.

When the toxicity 910 is selected, a first level of risk drug 920 is an actionable level having a very high side effect possibility, and is displayed with red. In addition, like the disease, a second step of risk drug is a significant level, and is displayed with an orange color. A third level of risk drug is a notable level and is displayed with yellow.

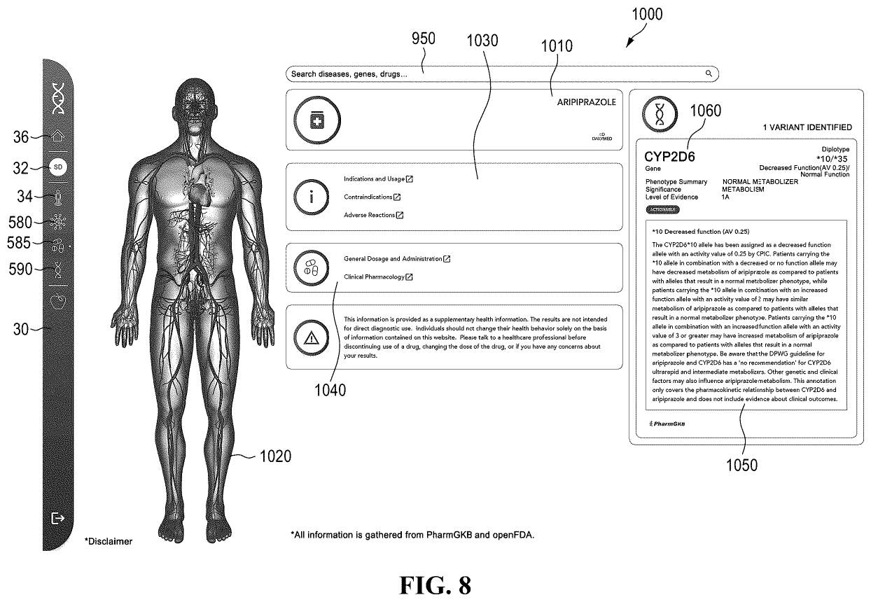

FIG. 8 is a diagram illustrating a detailed drug informatics unit 1000 displayed by clicking one drug of FIG. 7 according to an example embodiment of the present disclosure. As illustrated in FIG. 8, an integrated search unit 950 is provided at the top of the detailed drug information unit 1000, and the menu bar 30 and the 3D human body avatar 1020 are provided at the left side. In the detailed drug information unit 1000, a drug name 1010, indication and usage, contraindication, and adverse reaction 1030, general dosage and administration 1040, detailed information 1050, and a gene's name 1060 are displayed. At one side, a 3D human body avatar 1020 is displayed and a site (for example, liver) for the drug or the disease is displayed in blinking. The 3D human body avatar 1020 may be used as a graphic model of the digital twin 230.

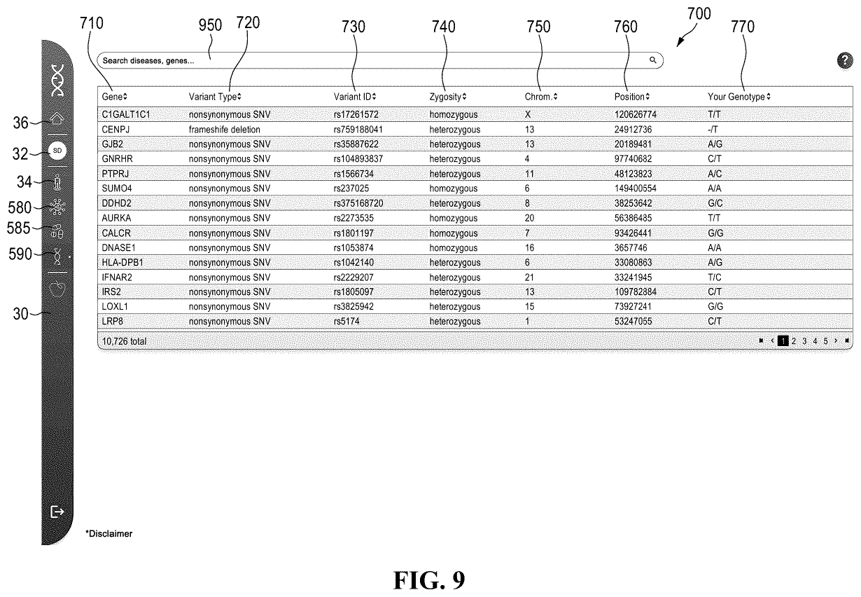

FIG. 9 is a diagram illustrating a genetic variant list 700 displayed by a genetic information icon 590 of a system according to an example embodiment of the present disclosure. As illustrated in FIG. 9, the genetic variant list 700 includes fields of a name of a gene 710, a variant type 720, a variant ID 730, a zygosity type 740, a chromosome number 750, a position 760, and a genotype 770.

The name of a gene 710 may be F5, PRKCQ, GJB2, IL4R, and the like, and a total of 10630 genes are provided. The variant type 720 may be nonsynonymous SNV, frameshift deletion, and the like. The variant ID 730 may be rs6025, rs2236379, rs80338939, and the like. The zygosity type 740 may be heterozygous and homozygous, and the like. The chromosome number 750 may be 1, 10, 13, 16, and the like. The gene position 760 may be 169549811, 6485181, 20189547, 27344882, and the like. The genotype 770 may be cytosine C/thymine T, guanine G/adenine A, A/G, A/A, and the like.

As described above, the detailed description of the exemplary embodiments of the disclosed present disclosure is provided such that those skilled in the art implement and carry out the present disclosure. While the disclosure has been described with reference to the preferred embodiments, it will be understood by those skilled in the art that various changes and modifications of the present disclosure may be made without departing from the spirit and scope of the disclosure. For example, those skilled in the art may use configurations disclosed in the above-described exemplary embodiments by combining them with each other. Therefore, the present disclosure is not intended to be limited to the above-described exemplary embodiments but to assign the widest scope consistent with disclosed principles and novel features.

The present disclosure may be implemented in another specific form within the scope without departing from the spirit and essential feature of the present disclosure. Therefore, the detailed description should not restrictively be analyzed in all aspects and should be exemplarily considered. The scope of the present disclosure should be determined by rational interpretation of the appended claims and all changes are included in the scope of the present disclosure within the equivalent scope of the present disclosure. The present disclosure is not intended to be limited to the above-described exemplary embodiments but to assign the widest scope consistent with disclosed principles and novel features. Further, claims having no clear quoting relation in the claims are combined to configure the embodiment or may be included as new claims by correction after application.

### INDUSTRIAL APPLICABILITY−>DESCRIPTION OF MAIN REFERENCE NUMERALS OF DRAWINGS

10: Login screen
12: Email input window
14: Password input window
16: Login button
20: Dashboard screen
21: Patient name tab
22: Disease tab
24: Drug tab
25: Search keyword input window
26: Search button
28: Patient name
30: Menu bar
32: Patient icon
34: Digital twin icon
36: Home icon
40: Patient information screen
41: Name
42: Biological sex
43: Birth data
44: Phone number
45: Address
46: Input button
100: DNA sequencing analysis unit
110: Sequencing data
200: Digital twin predicting unit
214: Predicted candidate screening AI unit
216: Genetic informatics pipeline unit
218: Dimensionality reduction AI unit
220: Genetic informatics integration unit
230: Digital twin file
240: Disease information table
250: Drug information table
260: Controller
270: Security checking unit
300: Public database
310: CLINVAR database
320: MedGen database
400: Service unit
410: External application
420: Dedicated Application
430: Report
500: Main screen
510: Medical record part
520: Disease prediction part
530: Drug side effect prediction part
540: Document part
545: Document creation button
560: Chatting part
570: Memo pad
580: Susceptible disease icon
585: Drug icon
590: Genetic information icon
700: Genetic variant list
710: Gene
720: Variant type
730: Variant ID
740: Zygosity type
750: Chromosome
760: Position
770: Genotype
800: Susceptible disease predicting unit
810, 820, 830, 840: First, second, third, and fourth susceptible diseases
850: Detailed disease informatics unit
851: Disease name
852: Variant gene name
853: DNA variant
854: Clinical significance
855: Disease information
856: Review state
858: Position and detailed information
900: Drug risk predicting unit
910: Toxicity
921: Dosage
914: Efficacy
916: Metabolism
920: First level of risk drug
950: Integrated search unit
1000: Detailed drug informatics unit
1010: Drug name
1020: 3D human avatar
1030: Usage, contraindications, and adverse reaction
1040: General dosage and administration
1050: Detailed drug information
1060: Gene's name

1. A disease or drug association providing system for a digital twin with genetic information screened by an artificial intelligence, the system comprising:
a clinical information generating unit including:
(i-1) a genetic informatics pipeline unit which sequentially outputs genetic sequencing data of a patient in a predetermined unit;
(i-2) a predicted candidate group screening AI unit which screens a predicted candidate group for the disease and a predicted candidate group for the drug with respect to the genetic information output from the genetic informatics pipeline unit;
(i-3) a dimensionality reduction AI unit which reduces a size of the genetic information; and
(i-4) a genetic informatics integration unit which receives and integrates the genetic information trained by the predicted candidate group screening AI unit and the dimensionality reduction AI unit from the genetic informatics pipeline unit;
(ii) a patient's digital twin file which is virtually generated based on information about a body of the patient, and the predicted candidate group for the disease and the predicted candidate group for the drug received from the genetic informatics integration unit;
(iii) a disease information table which receives and stores information about a disease from the outside;
(iv) a drug information table which receives and stores information about a drug from the outside; and
(v) a controller which calculates and provides an association with the disease information table about the genetic information of the digital twin file and an association with the drug information table about the genetic information of the digital twin file.

2. The disease or drug association providing system for a digital twin with genetic information screened by an artificial intelligence of claim 1, wherein the association of the digital twin file with the disease information table for the genetic information is calculated based on a variant gene name and a disease name among the genetic information.

3. The disease or drug association providing system for a digital twin with genetic information screened by an artificial intelligence of claim 1, wherein the association of the digital twin file with the drug information table for the genetic information is calculated based on the variant gene name and a drug name of the genetic information.

4. The disease or drug association providing system for a digital twin with genetic information screened by an artificial intelligence of claim 1, wherein the association of the digital twin file with the disease information table for the genetic information or the association of the digital twin file with the drug information table for the genetic information calculated by the controller is provided to an external service unit via a network.

5. The disease or drug association providing system for a digital twin with genetic information screened by an artificial intelligence of claim 4, wherein the service unit is an external application program of a client terminal which is connected to the network or a dedicated application program which is directly connected to the controller via the network.

6. The disease or drug association providing system for a digital twin with genetic information screened by an artificial intelligence of claim 5, wherein the external application program or the dedicated application program outputs a report including the association of the digital twin file with the disease information table for the genetic information or the association of the digital twin file with the drug information table for the genetic information.

7. The disease or drug association providing system for a digital twin with genetic information screened by an artificial intelligence of claim 6, wherein the report further includes a detailed disease informatics unit for the disease and a detailed drug informatics unit for the drug.

8. The disease or drug association providing system for a digital twin with genetic information screened by an artificial intelligence of claim 1, wherein the digital twin file further includes a 3D human body avatar model.

9. The disease or drug association providing system for a digital twin with genetic information screened by an artificial intelligence of claim 8, wherein a body part for the disease having the highest association or a body part for the drug having the highest association is graphically displayed on the 3D human body avatar model.

10. The disease or drug association providing system for a digital twin with genetic information screened by an artificial intelligence of claim 1, wherein the dimensionality reduction AI unit reduces the genetic information in a range of 20000.
